(19) United States
(12) Patent Application Publication
(10) Pub. No.: US 2022/0257161 A1
(43) Pub. Date: Aug. 18, 2022

(54) TESTING METHOD, SYSTEM AND DEVICE FOR FLICKER FUSION FREQUENCY RANGE

(71) Applicants: China National Institute of Standardization, Beijing (CN); Capital Normal University, Beijing (CN)

(72) Inventors: Yunhong ZHANG, Beijing (CN); Jinhong DING, Beijing (CN); Yuan LV, Beijing (CN); Chaoyi ZHAO, Beijing (CN); Taijie LIU, Beijing (CN); Changbing HUANG, Beijing (CN); Feng SI, Beijing (CN); Wei LI, Beijing (CN); Yongjia SHEN, Beijing (CN)

(73) Assignees: China National Institute of Standardization, Beijing (CN); Capital Normal University, Beijing (CN)

(21) Appl. No.: 17/733,633

(22) Filed: Apr. 29, 2022

Related U.S. Application Data
(63) Continuation of application No. PCT/CN2020/125959, filed on Nov. 2, 2020.

(30) Foreign Application Priority Data
Nov. 1, 2019 (CN) 201911060086.6

(51) Int. Cl. A61B 5/16; H05B 39/09; A61B 3/00

(52) U.S. Cl. CPC A61B 5/161; A61B 3/0008; H05B 39/09

(57) ABSTRACT

Disclosed are a testing method, a system and a device for flicker fusion frequency range. The testing method comprises: taking end point values of a self-preset range as starting judgment values; acquiring a flicker judgment result of a subject; if the flicker judgment result is flickering, continuing to execute the acquisition step by increasing a judgement value until the flicker judgment result turns into non-flickering, and defining a judgement value at the moment of turning as an judgment valueᵤₚ; if the flicker judgment result is non-flickering, continuing to execute the acquisition step by decreasing a judgement value until the flicker judgment result turns into flickering, and defining a judgement value at the moment of turning as an judgment value_{down}; and acquiring a flicker fusion frequency range which takes a judgment valueᵤₚ and a judgment value_{down} as end point values.

### CROSS-REFERENCE TO RELATED APPLICATIONS

The present application is a continuation of International Application No. PCT/CN2020/125959, filed on Nov. 2, 2020, which claims priority to Chinese Patent Application No. 201911060086.6, filed on Nov. 1, 2019. The contents of the above-mentioned applications are incorporated herein by reference in their entireties.

### TECHNICAL FIELD

The present application relates to the field of the testing technology for flicker fusion frequency, in particular to a testing method, a system and a device for flicker fusion frequency range.

### BACKGROUND

The so-called flicker fusion means that when a series of flickers come into human eyes, the eyes no longer feel the flickers but a fixed or continuous light if the flickering number per second increases to a certain level. In vision, this phenomenon is called flicker fusion. The critical flicker frequency (CFF) refers to the minimum frequency of the stimulation that can just cause the sensation of flicker fusion, represents the extreme limit of the capability of a visual system for resolving time, is an indicator of the capability of human eyes for time resolution of light stimulation, is a result of the interaction between physical stimulation and physiological and psychological functions, and is a sensory process of restriction between stimulated time and space factors and the body state. Usually, we use the limit method to measure the minimum frequency that causes the sensory stimulation of flicker fusion. Thus, the flicker fusion frequency meter is a key instrument for this kind of experiments.

The traditional flicker fusion frequency meter is generally used to measure the critical flicker frequency with a limit method, and the specific experimental process is as follows:

Increasing series experiments: the experimenter adjusts a light spot to flicker distinctly, and then reads the instruction: “what you see is a flickering spot, please turn the knob until you just can't see the light spot flickering; repeatedly adjust between flickering and non-flickering until you have confirmed that the light spot is no longer flickering, and then, please report to the experimenter”. The experimenter records this frequency value.

Decreasing series experiments: the experimenter adjusts a light spot until it is non-flickering distinctly, and then reads the instruction: “what you see is a non-flickering spot, please turn the knob until you just can see the light spot flickering; repeatedly adjust between flickering and non-flickering until you have confirmed that the light spot is flickering, and then, please report to the experimenter”. The experimenter records this frequency value.

There are two types of system errors in measurement of the flicker fusion frequency using the traditional flicker fusion frequency meter and the limit method, wherein the first type of system errors includes habitual error and expected error; and the second type of system errors includes practice error and fatigue error, specifically comprising:

Habitual error: it is manifested that the subject still reports that he cannot feel the stimulation in an increasing sequence even if the stimulation intensity has already exceeded the threshold due to response bias caused by habituation to the previous stimulations.

Expected error: contrary to the habitual error, the threshold will be lower when the subject gives an opposite judgment in a long sequence, and the threshold will be lower in the measurement of a decreasing sequence. The error caused by habits and expectations is a unique error of the limit method.

Practice error: it is a systematic error that the response is sped up and the accuracy is improved due to a case that the subject is getting familiar with the experimental scene or has interests and learning effects in the experiment after multiple repetitions of the experiment.

Fatigue error: it is a systematic error that the response speed and the accuracy are gradually reduced due to tiredness or boredom after repeated experiments.

In order to overcome the above systematic errors, the method used is to alternately present the increasing and decreasing sequences in an order of ABBA using the traditional flicker fusion frequency meter, wherein the increasing and decreasing sequences are used equally and have the equal chances of the first in the whole sequence. In such a way, even if there is practice effect or fatigue effect during the whole experiment, the effects will act on the increasing and decreasing sequences on average without additional interference. However, the experimental efficiency of the method is very low, because the experimenter does not know the flicker fusion frequency of each subject in advance and needs to take a lot of time to set the initial frequency and the flicker fusion frequency range, experimental data is recorded manually and the experimental results cannot be automatically recorded and calculated based on the experimental data.

### SUMMARY

In order to solve at least one of the above problems, the present application provides a testing method for flicker fusion frequency range. The present application further provides a controller, a testing system for flicker fusion frequency range, electronic equipment, and a non-transitory computer-readable storage medium.

A testing method for flicker fusion frequency range comprises:

starting step: taking end point values of a self-preset range as starting judgment values;

acquisition step: acquiring a flicker judgment result of the judgement value, the flicker judgment result comprising flickering or non-flickering; and the end point values comprising an upper limit of a frequency flickering distinctly and/or a lower limit of a frequency non-flickering distinctly;

adapting step: if the flicker judgment result is flickering, continuously executing the acquisition step by increasing a judgement value until the flicker judgment result turns into non-flickering, and defining a judgement value at the moment of turning as an judgment valueᵤₚ; if the flicker judgment result is non-flickering, continuously executing the acquisition step by decreasing a judgement value until the flicker judgment result turns into flickering, and defining a judgement value at the moment of turning as an judgment value_{down}; and

taking a judgment valueᵤₚ and a judgment value_{down} acquired in the nth time as end point values, thereby acquiring a flicker fusion frequency range, where n is greater than or equal to 1.

Optionally, if the flicker judgment result is flickering, continuously executing the acquisition step by increasing a judgement value until the flicker judgment result turns into non-flickering, and defining a judgement value at the moment of turning as a judgment valueᵤₚ comprises:

if the flicker judgement result is flickering, determining whether the flicker judgement result acquired this time is consistent to the flicker judgement result acquired last time;

if the results are consistent, increasing the judgment value at a stepping frequency, and executing the acquisition step again; and

if the results are inconsistent, recording a judgment value acquired this time and defining as a judgment valueᵤₚ, increasing the judgment value at a first preset frequency, and then executing the acquisition step.

Optionally, if the flicker judgment result is non-flickering, continuously executing the acquisition step by decreasing a judgement value until the flicker judgment result turns into flickering, and defining a judgement value at the moment of turning as a judgment value_{down} comprises:

if the flicker judgement result is non-flickering, determining whether the flicker judgement result acquired this time is consistent to the flicker judgement result acquired last time;

if the results are consistent, decreasing the judgment value at a stepping frequency, and executing the acquisition step again;

if the results are inconsistent, recording a judgment value acquired this time and defining as a judgment value_{down}, increasing the judgment value at a second preset frequency, and then executing the acquisition step.

Optionally, the first preset frequency ranges from 0 to 6 Hz.

Optionally, the second preset frequency ranges from 0 to 6 Hz.

Optionally, the stepping frequency ranges from 1 to 3 Hz.

The application also provides a controller, comprising a judgement module and an acquisition module,

the judgment module being used for determining a judgment value, and starting judgment values being end point values of a preset range; the judgment module being further used for determining a judgment value in each testing according to a flicker judgment result acquired by the acquisition module, if the flicker judgment result is flickering, continuing to execute the acquisition step in a manner of increasing the judgment value until the flicker judgment result turns into non-flickering, and defining a judgment value at the moment of turning as a judgment valueᵤₚ; if the flicker judgment result is non-flickering, continuing to execute the acquisition step in a manner of decreasing the judgment value until the flicker judgment result turns into flickering, and defining a judgment value at the moment of turning as a judgment value_{down}; and taking a judgment valueᵤₚ and a judgment value_{down} acquired in the nth time as end point values, thereby acquiring a flicker fusion frequency range, where n is greater than or equal to 1; and

the acquisition module being used for acquiring a flicker judgement result of the judgement value, the flicker judgement result comprising flickering or non-flickering, and the end point values of the preset range comprising an upper limit of a frequency flickering distinctly and/or a lower limit of a frequency non-flickering distinctly.

The application also provides a testing system for flicker fusion frequency range, comprising a flicker fusion frequency meter and the controller, and the controller being connected with the flicker fusion frequency meter.

The application also provides an electronic equipment, comprising:

at least one processor, at least one memory, a communication interface and a bus, wherein the processor, the memory, and the communication interface being communicated with one another through the bus; and the memory storing program instructions executed by the processor; and the processor calling the program instructions to execute the testing method.

A non-transitory computer-readable storage medium stores computer instructions which make the computer execute the testing method.

In the technical solution provided by the present application, taking the end points of a frequency range from distinctly flickering to non-flickering distinctly as starting points, increasing or decreasing a stepping frequency to change a judgment value, acquiring a flicker judgment result of the subject, extracting a judgment value corresponding to a point that the flicker judgment result changes, decreasing or increasing a stepping frequency in an opposite direction to acquire a judgment value corresponding to a point that the flicker judgment result changes, and acquiring a judgment valueᵤₚ and a judgment value_{down} acquired in the nth time after n repetitions back and forth, thereby constructing a flicker fusion frequency range. According to the method, an adaptive method is used for round testing, the stepping frequency is automatically adjusted, and practice error and fatigue effect are fundamentally eliminated. When n is greater than 1, habitual error and expected effect are eliminated by a testing sequence matched in multiple rounds of testing; thus, the testing accuracy is greatly improved and the time cost for testing is saved.

### BRIEF DESCRIPTION OF DRAWINGS

FIG. 1 is a schematic diagram of a testing method for flicker fusion frequency range in an embodiment of the present application.

### DETAILED DESCRIPTION OF EMBODIMENTS

The embodiments of the present disclosure are described in detail below. Examples of the embodiments are illustrated in the accompanying drawings, throughout which the same or similar reference numerals indicate the same or similar elements or elements with the same or similar functions. The embodiments described below with reference to the accompanying drawings are exemplary, and are intended to explain the present disclosure, but should not be understood as a limitation to the present disclosure.

Hereafter, the specific implementation of the present application will be further described in detail in conjunction with the embodiments.

In the description of the present application, it should be noted that, unless otherwise specified, the terms “installation”, “connected” and “connection” should be understood in a broad sense, for example, it may be a fixed connection, a detachable connection or an integral connection, or a mechanical connection or an electrical connection, or a direct connection or an indirect connection by an intermediate medium, or an internal communication between two components. The terms “first”, “second”, “third”, and “fourth” do not represent any sequence relationship, and are only for ease of distinguishing. Those of ordinary skill in the art may understand the specific meanings of the above-mentioned terms in the present application under specific circumstances. In the text, “up” and “down” are defined in terms of the specific position of the product in use.

In order to solve the problems that the current testing method for flicker fusion frequency range has large errors and inaccurate results, the present application provides a testing method, a testing system and a device for flicker fusion frequency range.

Hereinafter, the products and methods will be described in detail through basic designs, extension designs and alternative designs.

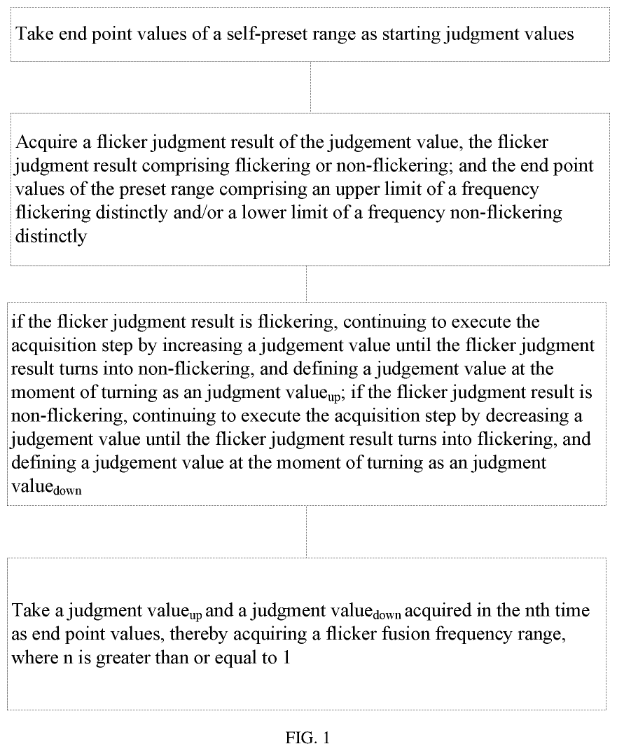

The present application provides a testing method for flicker fusion frequency range. A testing method for flicker fusion frequency range, comprising, As illustrated in FIG. 1:

starting step: taking end point values of a self-preset range as starting judgment values;

acquisition step: acquiring a flicker judgment result of the judgement value, the flicker judgment result comprising flickering or non-flickering; and the end point values comprising an upper limit of a frequency flickering distinctly and/or a lower limit of a frequency non-flickering distinctly;

adapting step: if the flicker judgment result is flickering, continuously executing the acquisition step by increasing a judgement value until the flicker judgment result turns into non-flickering, and defining a judgement value at the moment of turning as an judgment valueᵤₚ; if the flicker judgment result is non-flickering, continuously executing the acquisition step by decreasing a judgement value until the flicker judgment result turns into flickering, and defining a judgement value at the moment of turning as an judgment value_{down}; and

taking a judgment valueᵤₚ and a judgment value_{down} acquired in the nth time as end point values, thereby acquiring a flicker fusion frequency range, where n is greater than or equal to 1.

The preset range is from an upper limit of a frequency flickering distinctly to a lower limit of a frequency non-flickering distinctly, and those skilled in the art may set it according to empirical values. The preset range is to give a large range value, for example, 20 to 90 Hz. The frequency defining method by “distinct” is that one normal subject judges flickering ad non-flickering directly and unambiguously, that is to say, the defining method is suitable for all subjects with normal vision.

The stepping frequency can guarantee regular and gradual adjustments during the rounds of testing, to improve the testing accuracy. A judgment value is adjusted at a stepping frequency to achieve automatic adjustment, thereby avoiding inaccuracy and fatigue error caused by manual and autonomous adjustments. The stepping frequency is preferably 1 to 3 Hz, more preferably 1 Hz.

When n is 1, it is equivalent to one round of testing. When n is more than one, it is equivalent to multiple testing repetitions. When the nth testing is performed, the judgment valueᵤₚ and the judgment value_{down} in the nth testing are used to construct a flicker fusion frequency testing range.

In the technical solution provided by the present application, taking the end points of a frequency range from distinctly flickering to non-flickering distinctly as starting points, increasing or decreasing a stepping frequency to change a judgment value, acquiring a flicker judgment result of the subject, extracting a judgment value corresponding to a point that the flicker judgment result changes, decreasing or increasing a stepping frequency in an opposite direction to acquire a judgment value corresponding to a point that the flicker judgment result changes, and acquiring a judgment valueᵤₚ and a judgment value_{down} acquired in the nth time after n repetitions back and forth, thereby constructing a flicker fusion frequency range. According to the method, an adaptive method is used for round testing, the stepping frequency is automatically adjusted, and practice error and fatigue effect are fundamentally eliminated. When n is greater than 1, habitual error and expected effect are eliminated by a testing sequence matched in multiple rounds of testing; thus, the testing accuracy is greatly improved and the time cost for testing is saved.

It should be noted that, after the judgment valueᵤₚ is acquired, in order to improve the testing accuracy, the judgment value of the next testing may be increased by a first preset frequency on the basis of the judgment valueᵤₚ, that is, the judgment value is increased based on the judgment valueᵤₚ, and then the judgment value is decreased at the stepping frequency to test in turn. Similarly, after the judgment value_{down} is acquired, in order to improve the testing accuracy, the judgment value of the next testing may be he flicker judgment result is non-flickeringd by a second preset frequency on the basis of the judgment value_{down}, that is, the judgment value is reduced based on the judgment value_{down}, and then the judgment value is increased at the stepping frequency to test in turn. This method can improve the testing accuracy and avoid practice error and fatigue effect caused by visual fatigue of the subject. The first preset frequency is 0 to 6 Hz, preferably 5 Hz. The second preset frequency is 0 to 6 Hz, preferably 5 Hz.

Specific embodiments are given below.

### Embodiment 1

First, determining a preset range (from an upper limit of a frequency flickering distinctly to a lower limit of a frequency non-flickering distinctly) and a stepping frequency.

Then, taking the upper limit of a frequency flickering distinctly as a starting judgment value, wherein a subject sees a flashpoint of the upper limit of a frequency flickering distinctly first and then determines whether this point is flickering, thereby getting a flicker judgment result.

If the subject determines that it is a “flickering”, acquiring the flicker judgment result; next, increasing the stepping frequency on the basis of the original judgment value to form a new judgment value; and then, the subject sees a flashpoint of the new judgement value and acquires a flicker judgment result by determining weather this point is flickering; if the flicker judgment result is still “flickering”, continuing to increase a stepping frequency on the basis of the last flicker judgment result for testing until a flicker judgment result given by the subject is “non-flickering”; and recording a judgment value corresponding to “non-flickering” and defining as a judgment valueᵤₚ.

On the basis of the judgment valueᵤₚ, increasing the preset frequency for testing again; if the flicker judgment result acquired is “non-flickering”, decreasing the stepping frequency from the last judgment value for testing until a flicker judgment result given by the subject is “flickering”; recording a judgment value corresponding to “flickering” and defining as a judgment value_{down}.

Consequently, (judgment valueᵤₚ, judgment value_{down}) is a flicker fusion frequency range.

Definitely, a second round of testing may also be performed in other embodiments. If performing the second round of testing, decreasing the preset frequency on the basis of the “judgment value_{down}” for testing again; if the flicker judgment result acquired is “flickering”, increasing the stepping frequency from the last judgment value. The testing is continuously performed step by step, like the first round of the testing, until a judgment value_{down} and a judgment valueᵤₚ in the second time are acquired. The values in the second time are used for determining the flicker fusion frequency range.

Moreover, in other embodiments, the third round, the fourth round, or more rounds of testing may be performed; the method is similar to that in the second round of testing; and the flicker fusion frequency range can be determined based on a judgment value_{down} and a judgment valueᵤₚ acquired last time.

### Embodiment 2

First, determining a preset range (from an upper limit of a frequency flickering distinctly to a lower limit of a frequency non-flickering distinctly) and a stepping frequency.

Then, taking the lower limit of a frequency flickering distinctly as a starting judgment value, wherein a subject sees a flashpoint of the lower limit of a frequency flickering distinctly first and then determines whether this point is flickering, thereby getting a flicker judgment result.

If the subject determines that it is “non-flickering”, acquiring the flicker judgment result; next, decreasing the stepping frequency on the basis of the original judgment value to form a new judgment value; and then, the subject sees a flashpoint of the new judgement value and acquires a flicker judgment result by determining weather this point is flickering; if the flicker judgment result is still “non-flickering”, decreasing a stepping frequency on the basis of the last flicker judgment result for testing until a flicker judgment result given by the subject is “flickering”; and recording a judgment value corresponding to “flickering” and defining as a judgment value_{down}.

On the basis of the judgment value_{down}, decreasing the preset frequency to perform a test again; if the flicker judgment result acquired is “flickering”, increasing the stepping frequency from the last judgment value. The test is continued until a flicker judgment result given by the subject is “non-flickering”; and recording a judgment value corresponding to “non-flickering” and defining as a judgment valueᵤₚ.

Consequently, (judgment value_{down}, judgment valueᵤₚ) is a flicker fusion frequency range.

Definitely, a second round of testing may also be performed in other embodiments. If performing the second round of testing, increasing the preset frequency on the basis of the “judgment valueᵤₚ” for testing again; if the flicker judgment result acquired is “non-flickering”, decreasing the stepping frequency from the last judgment value. The test is continuously performed step by step, like the first round of the testing, until a judgment value_{down} and a judgment valueᵤₚ in the second time are acquired. The values in the second time are used for determining the flicker fusion frequency range.

Moreover, in other embodiments, the third round, the fourth round, or more rounds of testing may be performed; the method is similar to that in the second round of testing; and the flicker fusion frequency range can be determined based on a judgment value_{down} and a judgment valueᵤₚ acquired last time.

### Embodiment 3

Embodiment 3 is a more specific implementation mode of embodiment 1.

First, setting a frequency range from flickering distinctly to non-flickering distinctly, for example, 20 to 90 Hz, and a stepping frequency, for example, 1 Hz; and then letting a subject to see a flashpoint of 20 Hz and to determine whether this point is flickering, if the subject determines that it is “flickering”, the system will automatically increase the flicker frequency by 1 Hz to reach 21 Hz; if the subject still determines that it is “flickering”, continuously increasing the flicker frequency until it reaches a frequency (such as 55 Hz) at which the subject determines that the point is non-flickering, then the system increases the frequency by 5 Hz on the above basis, that is, decreasing by 1 Hz after reaching 60 Hz until it drops to a frequency (for example, 30 Hz) at which the subject selects “flickering”; next, decreasing the frequency by 5 Hz, that is, increasing the frequency again for selection after 25 Hz. In this way, a flicker fusion variation range (from 25 Hz to 60 Hz) of a specific subject can be automatically determined through a process of increasing and decreasing. On this basis, letting the subject to perform more than two rounds of testing, and finally automatically calculating a flicker fusion frequency of the subject according to the response data of all rounds of testing.

The present application provides a controller, comprising a judgement module and an acquisition module, the judgment module being used for determining a judgment value, and starting judgment values being end point values of a preset range; the judgment module being further used for determining a judgment value in each testing according to a flicker judgment result acquired by the acquisition module, if the flicker judgment result is flickering, continuing to execute the acquisition step in a manner of increasing the judgment value until the flicker judgment result turns into non-flickering, and defining a judgment value at the moment of turning as a judgment valueᵤₚ; if the flicker judgment result is non-flickering, continuing to execute the acquisition step in a manner of decreasing the judgment value until the flicker judgment result turns into flickering, and defining a judgment value at the moment of turning as a judgment value_{down}; and taking a judgment valueᵤₚ and a judgment value_{down} acquired in the nth time as end point values, thereby acquiring a flicker fusion frequency range, where n is greater than or equal to 1; and

the acquisition module being used for acquiring a flicker judgement result of the judgement value, the flicker judgement result comprising flickering or non-flickering, and the end point values of the preset range comprising an upper limit of a frequency flickering distinctly and/or a lower limit of a frequency non-flickering distinctly.

The controller may be a single-chip microcomputer. In the technical solution provided by the present application, taking the end points of a frequency range from distinctly flickering to non-flickering distinctly as starting points, increasing or decreasing a stepping frequency to change a judgment value, acquiring a flicker judgment result of the subject, extracting a judgment value corresponding to a point that the flicker judgment result changes, decreasing or increasing a stepping frequency in an opposite direction to acquire a judgment value corresponding to a point that the flicker judgment result changes, and acquiring a judgment valueᵤₚ and a judgment value_{down} acquired in the nth time after n repetitions back and forth, thereby constructing a flicker fusion frequency range. According to the method, an adaptive method is used for round testing, the stepping frequency is automatically adjusted, and practice error and fatigue effect are fundamentally eliminated. When n is greater than 1, habitual error and expected effect are eliminated by a testing sequence matched in multiple rounds of testing; thus, the testing accuracy is greatly improved and the time cost for testing is saved.

The present application further provides a testing system for flicker fusion frequency range, comprising: a flicker fusion frequency meter and a controller; the controller is connected with the flicker fusion frequency meter; and the controller controls the frequency change of the flicker fusion frequency meter and the corresponding frequency of the flashpoint.

In the technical solution provided by the present application, taking the end points of a frequency range from distinctly flickering to non-flickering distinctly as starting points, increasing or decreasing a stepping frequency to change a judgment value, acquiring a flicker judgment result of the subject, extracting a judgment value corresponding to a point that the flicker judgment result changes, decreasing or increasing a stepping frequency in an opposite direction to acquire a judgment value corresponding to a point that the flicker judgment result changes, and acquiring a judgment valueᵤₚ and a judgment value_{down} acquired in the nth time after n repetitions back and forth, thereby constructing a flicker fusion frequency range. According to the method, an adaptive method is used for round testing, the stepping frequency is automatically adjusted, and practice error and fatigue effect are fundamentally eliminated. When n is greater than 1, habitual error and expected effect are eliminated by a testing sequence matched in multiple rounds of testing; thus, the testing accuracy is greatly improved and the time cost for testing is saved.

The present application further provides an electronic equipment, comprising: at least one processor, at least one memory, a communication interface and a bus, wherein the processor, the memory, and the communication interface are communicated with one another through the bus; the memory stores program instructions executed by the processor; and the processor calls the program instructions to execute the testing method.

In the technical solution provided by the present application, taking the end points of a frequency range from distinctly flickering to non-flickering distinctly as starting points, increasing or decreasing a stepping frequency to change a judgment value, acquiring a flicker judgment result of the subject, extracting a judgment value corresponding to a point that the flicker judgment result changes, decreasing or increasing a stepping frequency in an opposite direction to acquire a judgment value corresponding to a point that the flicker judgment result changes, and acquiring a judgment valueᵤₚ and a judgment value_{down} acquired in the nth time after n repetitions back and forth, thereby constructing a flicker fusion frequency range. According to the method, an adaptive method is used for round testing, the stepping frequency is automatically adjusted, and practice error and fatigue effect are fundamentally eliminated. When n is greater than 1, habitual error and expected effect are eliminated by a testing sequence matched in multiple rounds of testing; thus, the testing accuracy is greatly improved and the time cost for testing is saved.

The present application further provides a non-transitory computer-readable storage medium. The non-transitory computer-readable storage medium stores computer instructions which make the computer execute the testing method.

Any process or method description in the flowchart or described in other ways herein can be understood to include one or more modules, segments or portions of codes of executable instructions for achieving specific logic functions or steps in the process, and the scope of the preferred embodiments of the present disclosure includes additional implementations, which may implement functions in an order rather than the order shown or discussed, including implementing the functions in a substantially simultaneous manner or in a reverse order depending on the functions involved. This should be understood by those skilled in the art to which the embodiments of the present disclosure belong.

For example, the logic and/or steps represented in the flowchart or described in other manners herein can be considered as a sequenced list of executable instructions for implementing logic functions, and can be embodied in any computer-readable medium to be used by instruction execution systems, apparatuses, or devices (such as computer-based systems, systems including processors, or other systems that can read and execute instructions from instruction execution systems, apparatuses, or devices), or for use in combination with these instruction execution systems, apparatuses or devices. For the purposes of this specification, a “computer-readable medium” may be any device that can contain, store, communicate, propagate, or transmit a program to be used by instruction execution systems, apparatuses, or devices or in combination with these instruction execution systems, apparatuses, or devices. More specific examples (non-exhaustive list) of computer-readable media include: electrical connections (electronic devices) with one or more wirings, portable computer disk cases (magnetic devices), random access memory (RAM), read-only memory (ROM), erasable and editable read-only memory (EPROM or flash memory), optical fiber devices, and portable compact disk read-only memory (CDROM). In addition, the computer-readable medium may even be a paper or other appropriate media capable of printing programs thereon, because the program can be obtained electronically for example by optically scanning the paper or other media, and then editing, interpreting, or processing in other suitable manners if necessary, and then stored in the computer memory.

It should be understood that various parts of the present disclosure may be implemented by hardware, software, firmware, or a combination thereof. In the above embodiments, multiple steps or methods can be implemented by software or firmware stored in a memory and executed by a suitable instruction execution system. For example, if it is implemented by hardware as in another embodiment, it may be implemented by any one of or a combination of the following technologies known in the art: discrete logic circuits with logic gate circuits for implementing logic functions on data signals, application specific integrated circuits with suitable combinational logic gate circuits, programmable gate array (PGA), field programmable gate array (FPGA), etc.

It would be understood by those skilled in the art that all or part of the steps carried in the method of the foregoing embodiments may be implemented by a program instructing relevant hardware. The program may be stored in a computer-readable storage medium. When the program is executed, one or a combination of the steps of the method in the above-described embodiments may be completed.

In addition, the functions in the various embodiments of the present disclosure may be integrated into one processing module, or may be separately physically present, or two or more may be integrated into one module. The above-mentioned integrated modules may be implemented in the form of hardware, or may be implemented in the form of a software functional module. When the integrated module is implemented in the form of a software function module and sold or used as a separate product, it may also be stored in a computer readable storage medium.

The above-mentioned storage medium may be a read-only memory, a magnetic disk, or an optical disk, etc. Although the embodiments of the present disclosure have been shown and described above, it can be understood by those skilled in the art that the above-mentioned embodiments are exemplary and should not be construed as limiting the present disclosure, and changes, modifications, substitutions, and variations can be made in the foregoing embodiments without departing from scope of the present disclosure.

1. A testing method for flicker fusion frequency range, comprising:
starting step: taking end point values of a self-preset range as starting judgment values;
acquisition step: acquiring a flicker judgment result of the judgement value, the flicker judgment result comprising flickering or non-flickering; and the end point values comprising an upper limit of a frequency flickering distinctly and/or a lower limit of a frequency non-flickering distinctly;
adapting step: if the flicker judgment result is flickering, continuously executing the acquisition step by increasing a judgement value until the flicker judgment result turns into non-flickering, and defining a judgement value at the moment of turning as an judgment valueᵤₚ, and correcting the judgment value by adding a first preset frequency on the basis of the judgment valueᵤₚ when the flicker judgment result is non-flickering; if the flicker judgment result is non-flickering, continuously executing the acquisition step by decreasing a judgement value until the flicker judgment result turns into flickering, and defining a judgement value at the moment of turning as an judgment value_{down}, and correcting the judgment value by reducing a second preset frequency on the basis of the judgment value_{down} when the flicker judgment result is flickering; and
taking a judgment valueᵤₚ and a judgment value_{down} acquired in the nth time as end point values, thereby acquiring a flicker fusion frequency range, where n is greater than or equal to 1.

2. The testing method for flicker fusion frequency range of claim 1, wherein if the flicker judgment result is flickering, continuously executing the acquisition step by increasing a judgement value until the flicker judgment result turns into non-flickering, and defining a judgement value at the moment of turning as a judgment valueᵤₚ comprises:
if the flicker judgement result is flickering, determining whether the flicker judgement result acquired this time is consistent to the flicker judgement result acquired last time;
if the results are consistent, increasing the judgment value at a stepping frequency, and executing the acquisition step again; and
if the results are inconsistent, recording a judgment value acquired this time and defining as a judgment valueᵤₚ, increasing the judgment value at a first preset frequency, and then executing the acquisition step.

3. The testing method for flicker fusion frequency range of claim 1, wherein if the flicker judgment result is non-flickering, continuously executing the acquisition step by decreasing a judgement value until the flicker judgment result turns into flickering, and defining a judgement value at the moment of turning as a judgment value_{down} comprises:
if the flicker judgement result is non-flickering, determining whether the flicker judgement result acquired this time is consistent to the flicker judgement result acquired last time;
if the results are consistent, decreasing the judgment value at a stepping frequency, and executing the acquisition step again;
if the results are inconsistent, recording a judgment value acquired this time and defining as a judgment value_{down}, increasing the judgment value at a second preset frequency, and then executing the acquisition step.

4. The testing method for flicker fusion frequency range of claim 2, wherein the first preset frequency ranges from 0 to 6 Hz.

5. The testing method for flicker fusion frequency range of claim 3, wherein the second preset frequency ranges from 0 to 6 Hz.

6. The testing method for flicker fusion frequency range of claim 2, wherein the stepping frequency ranges from 1 to 3 Hz.

7. A controller, comprising a judgement module and an acquisition module,
the judgment module being used for determining a judgment value, and starting judgment values being end point values of a preset range; the judgment module being further used for determining a judgment value in each testing according to a flicker judgment result acquired by the acquisition module, if the flicker judgment result is flickering, continuing to execute the acquisition step in a manner of increasing the judgment value until the flicker judgment result turns into non-flickering, and defining a judgment value at the moment of turning as a judgment valueᵤₚ; if the flicker judgment result is non-flickering, continuing to execute the acquisition step in a manner of decreasing the judgment value until the flicker judgment result turns into flickering, and defining a judgment value at the moment of turning as a judgment value_{down}; and taking a judgment valueᵤₚ and a judgment value_{down} acquired in the nth time as end point values, thereby acquiring a flicker fusion frequency range, where n is greater than or equal to 1; and
the acquisition module being used for acquiring a flicker judgement result of the judgement value, the flicker judgement result comprising flickering or non-flickering, and the end point values of the preset range comprising an upper limit of a frequency flickering distinctly and/or a lower limit of a frequency non-flickering distinctly.

8. A testing system for flicker fusion frequency range, comprising a flicker fusion frequency meter and the controller of claim 7, and the controller being connected with the flicker fusion frequency meter.

9. An electronic equipment, comprising:
at least one processor, at least one memory, a communication interface and a bus, wherein
the processor, the memory, and the communication interface communicate with one another through the bus; and
the memory stores program instructions executed by the processor; and the processor calls the program instructions to execute the testing method of claim 1.

10. A non-transitory computer-readable storage medium, wherein the non-transitory computer-readable storage medium stores computer instructions which make the computer execute the testing method of claim 1.
